(19) United States
(12) Patent Application Publication
(10) Pub. No.: US 2007/0118928 A1
(43) Pub. Date: May 24, 2007

(54) Transgenic plant and method for altering oil and protein plant content

(76) Inventors: Yun Lin, Savoy, IL (US); Shui Wang, Chapel Hill, NC (US)

Correspondence Address: Jane Massey Licata;Licata & Tyrrell P.C., 66 E. Main Street, Marlton, NJ, 08053, US

(21) Appl. No.: 11/562,244

(22) Filed: Nov. 21, 2006

Related U.S. Application Data
(60) Provisional application No. 60/739,991, filed on Nov. 23, 2005.

(51) Int. Cl. A01H 1/00; C12N 15/82; C12N 5/04

(52) U.S. Cl. 800/281; 435/419; 435/468

(57) ABSTRACT

The present invention relates transgenic plants, progeny, seeds, and vegetatively reproducible structures of plants with reduced or elevated levels of ATP-phosphoribosyl transferase (ATP-PRT) expression or activity. Reduced or elevated levels of ATP-PRT have been found to alter protein or oil content in plants thereby modulating protein-to-oil ratios. Thus, the present invention also embraces a method for altering the protein or oil content of at least one tissue of a plant by modulating the expression or activity of ATP-PRT in the tissue.

### INTRODUCTION

This application claims the benefit of U.S. Provisional Application No. 60/739,991, filed Nov. 23, 2005, which is herein incorporated by reference in its entirety.

### BACKGROUND OF THE INVENTION

Living organisms adjust metabolism and development according to carbon, energy, and nitrogen status. The ability to sense organic nitrogen signals is crucial for the optimal survival and growth. Although twenty amino acids are metabolized in the cells, they do not serve equally as nitrogen signals. The signaling roles of glutamine and glutamate have been documented in microbial and implied in plants (Ninfa and Atkinson (2000) Trends Microbiol. 8:172-179; Coruzzi and Bush (2001) Plant Physiol. 125:61-64; Coruzzi and Zhou (2001) Curr. Opin. Plant Biol. 4:247-253). Besides these two amino acids, special regulatory functions appear to be associated with a third amino acid, histidine. Inhibition of histidine biosynthesis in Arabidopsis induces the transcripts of eight out of eleven amino acid biosynthetic genes and inhibited glutamine synthase (GS) expression (Guyer, et al. (1995) Proc. Natl. Acad. Sci. USA 92:4997-5000).

The biosynthesis of histidine consumes forty-one ATP molecules (Alifano, et al. (1996) Microbiol. Rev. 60:44-69). Biochemical pathways leading to histidine and de novo purine syntheses are linked; the two pathways share the same immediate precursor phosphoribosyl pyrophosphate (PRPP) and a by-product from the histidine pathway, 5-aminoimidazole-4-carboxamide ribonucleoside (AICAR), is an intermediate for purine biosynthesis. The close relationship of histidine and purine pathways is further reflected by the cross-regulation of the two pathways (Rebora, et al. (2Q01) Mol. Cell Biol. 21:7901-12; Rebora, et al. (2005) Genetics 170:61-70). Thus, histidine may serve as a nitrogen signal because it is integrated with energy metabolism.

The pathway of histidine biosynthesis is largely controlled by the first, committed step in which ATP-PRT catalyzes the condensation of PRPP and ATP to form phosphoriosyl-ATP (PR-ATP) (Alifino, et al. (1996) supra; Ohta, et al. (2000) Plant Physiol. 122:907-914). PRPP itself is a product of ribose-5 phosphate and ATP (Krath, et al. (1999) Biochim. Biophys. Acta 1430:403-408). Because ATP-PRT is allosterically and positively regulated by PRPP, negatively regulated by histidine and AMP, and its transcription is also subjected to negative-feedback by histidine, histidine is expected to be produced in balance with ribose-5 phosphate and ATP/AMP ratio.

PRPP stabilizes the active and homodimeric form of ATP-PRT. Binding of AMP favors the formation of the hexameric inactive form of the enzyme, which can be further stabilized by histidine. Crystal structures of ATP-PRT from Mycobacterium tuberculosis and Escherichia coli reveal three domains. Domains I and II form the catalytic core of the enzyme, whereas domain III is structurally similar to a PII signal transduction protein (Cho, et al. (2003) J. Biol. Chem. 278:8333-8339; Lohkamp, et al. (2004) J. Mol. Biol. 336:131-44). PII proteins are ancient signaling proteins that integrate the antagonizing signals of carbon and nitrogen status to control metabolism and growth. They regulate downstream events through PII receptor proteins and the interactions are sensitive to concentrations of carbon and nitrogen metabolites (Ninfa and Atkinson, (2000) supra; Moorhead and Smith (2003) Plant Physiol. 133:492-498). The competitive inhibitor AMP occupies both the PRPP and ATP binding sites at the catalytic domains of ATP-PRT, whereas the histidine interacting site is located in domain III.

The cDNAs corresponding to enzymes of all steps of histidine biosynthesis have been isolated from Arabidopsis or other species (Tada, et al. (1994) Plant Physiol. 105:579-583; Fujimori and Ohta (1998) Plant Physiol. 118:275-83; Fujimori and Ohta (1998) FEBS Lett. 428:229-34; Fujimori, et al. (1998) Mol. Gen. Genet. 259:216-223; Ohta, et al. (2000) supra; E1 Malki, et al. (1998) Plant Mol. Biol. 37:1013-1022; Nagai, et al. (1991) Proc. Natl. Acad. Sci. USA 88:4133-4137). There are two ATP-PRT isoforms in Arabidopsis, ATP-PRT1 and ATP-PRT2, both of which complement the yeast mutant that lacks the functional enzyme. In vitro studies further indicate that the plant and the microbial enzymes have similar enzymatic properties (Ohta, et al. (2000) supra). In addition, studies showed that ATP-PRT confers nickel tolerance by controlling the synthesis of histidine (Wycisk, et al. (2004) FEBS Lett. 578:128-134; Ingle, et al. (2005) Plant Cell. 17:2089-106).

### SUMMARY OF THE INVENTION

The present invention is a transgenic plant with reduced or elevated levels of ATP-phosphoribosyl transferase (ATP-PRT) expression or activity, wherein at least one tissue of the transgenic plant exhibits altered protein or oil content. Progeny, seeds, and vegetatively reproducible structures from the transgenic plant are also provided as is an expression vector containing a nucleic acid molecule encoding ATP-PRT operably linked to a plant promoter.

The present invention also embraces methods for altering the protein or oil content of at least one tissue of a plant by modulating the expression or activity of ATP-PRT in at least one tissue of the plant. In one embodiment, expression or activity is modulated by an agent. In another embodiment, expression or activity of ATP-PRT is modulated by introducing an expression vector containing a nucleic acid molecule encoding ATP-PRT and a plant promoter into a plant so that the expression or activity of ATP-phosphoribosyl transferase in at least one tissue of the plant is modulated.

### BRIEF DESCRIPTION OF THE DRAWINGS

FIG. 1 shows the weight of wild-type (wt) and atp-prt1-1 mutant seeds.

FIG. 2 shows the percent of seed weight attributed to oil and protein in wild-type (wt) and atp-prt1-1 mutant seeds.

FIG. 3 shows the metabolites present in atp-prt1-1 mutant seeds (FIG. 3A) and atp-prt1-1 mutant seedlings (FIG. 3B and FIG. 3C).

FIG. 4 shows real-time RT-PCR measurement of storage protein gene expression in 12 DAF (day after fertilization) developing seeds and mature dry seeds (Batch 1 and 2). At2S2 (left panel) and At2S4 (right panel) encode albumin isoforms. The expression of both genes was normalized to ubiquitin 10 (UBQ10).

FIG. 5 shows the fatty acid composition of atp-prt1-1 mutant and ATP-PRT1 transgenic seeds. Measurements of wild-type (WT), four F2 atp-prt1-1 mutant lines (a to d) and four ATP-PRT1 transgenic lines (OE1 to OE4) are shown in the order indicated. Bars indicate standard deviations.

### DETAILED DESCRIPTION OF THE INVENTION

It has now been found that ATP-PRT coordinates carbon and nitrogen metabolism thereby regulating seed protein-to-oil ratios. Not wishing to be bound by a particular theory, it is believed that histidine acts as a nitrogen signal to control multiple metabolic pathways and ATP-PRT plays a regulatory role by adjusting the flux of histidine biosynthesis according to carbon and nitrogen status. Thus, modulating ATP-PRT expression or activity is useful for maximizing seed oil or protein production and optimizing ratios of these products in seeds. In this regard, foods can be generated to contain particular levels of protein or oil to enhance nutrition or facilitate seed processing procedures.

To identify novel pathways and components involved in oil and protein storage, a genetic approach was taken to isolate mutants that exhibited altered oil or protein contents. A homozygous transgenic Arabidopsis line (A15) expressing a firefly luciferase reporter construct was generated. The expression cassette contained the Arabidopsis oil body protein oleosin (OLEO, At4g25140), fused in-frame with luciferase (LUC), and positioned between the 51 regulatory region of oleosin (Poleo) and the 3′ untranslated region of nopaline synthase (NOS 3′). Based on the topology of an OLEO-GUS fusion protein (van Rooijen and Moloney (1995) Plant Physiol. 109:1353-61), the OLEO-LUC fusion protein was expected to locate to the oil body membrane with the central hydrophobic region of oleosin embedded in the oil body and LUC exposed to the cytosol. Transgenic Arabidopsis lines expressing the OLEO-LUC protein exhibited luciferase activities in seeds and post-germinative seedlings but not in older plants, indicating that the expression of the fusion protein was seed-specific.

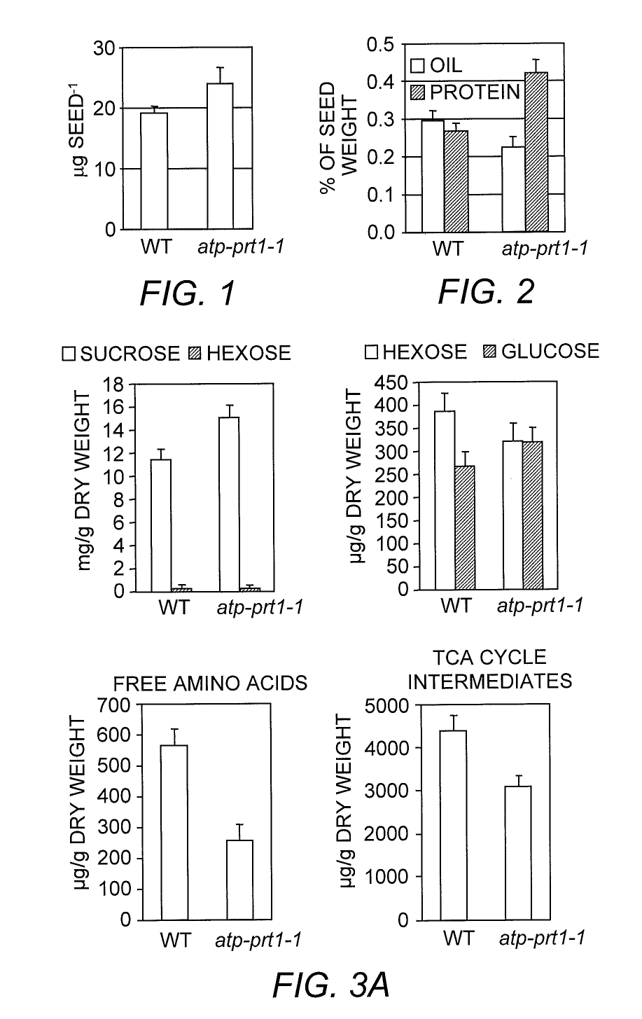

Mutants with elevated or reduced LUC activities were identified and analyzed for altered oil or protein content. As oleosin and storage protein genes are co-regulated in seeds (Ruuska, et al. (2002) Plant Cell 14:1191-1206), LUC activity reflected protein content. Moreover, because oleosin is stabilized on the oil body membrane, LUC activity was also influenced by oil content (van Rooijen and Moloney (1995) supra). A carbon partitioning mutant was selected from an EMS-mutagenized population. The mutant exhibited an increased protein-to-oil ratio; oil content was reduced to 22%, compared to 30% in the wild-type, and total protein content increased to 40%, compared to 27% in the wild-type plant. The mutant seeds were slightly heavier than the wild type seeds (FIG. 1 and FIG. 2). Because the seed phenotype was indicative of a fundamental function of the corresponding gene in regulating carbon partitioning between oil and protein, this mutant was further analyzed.

The gene was cloned by positional cloning and found to correspond to ATP-PRT1 (GENBANK Accession No. NP_{—}176105). A missense mutation had changed the conserved glycine residue at position 170 into glutamate. The atp-prt1-1 mutant plants were thinner than the wild-type with a severe defect in root growth. Both defects were rescued by adding histidine to the growth medium. Because null mutants obtained from SALK lines could not grow beyond early seedling stage without external histidine, the EMS-induced mutant was indicative of a leaky mutant. Null mutant lethality has also been shown for another histidine biosynthetic gene, BBMII isomerase (Noutoshi, et al. (2005) Plant Cell Physiol. 46(7):1165-72).

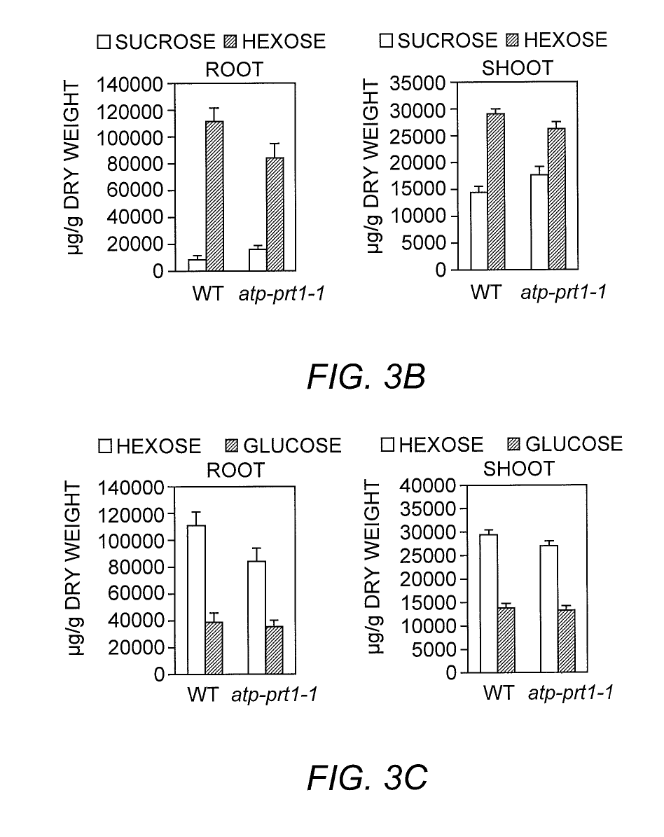

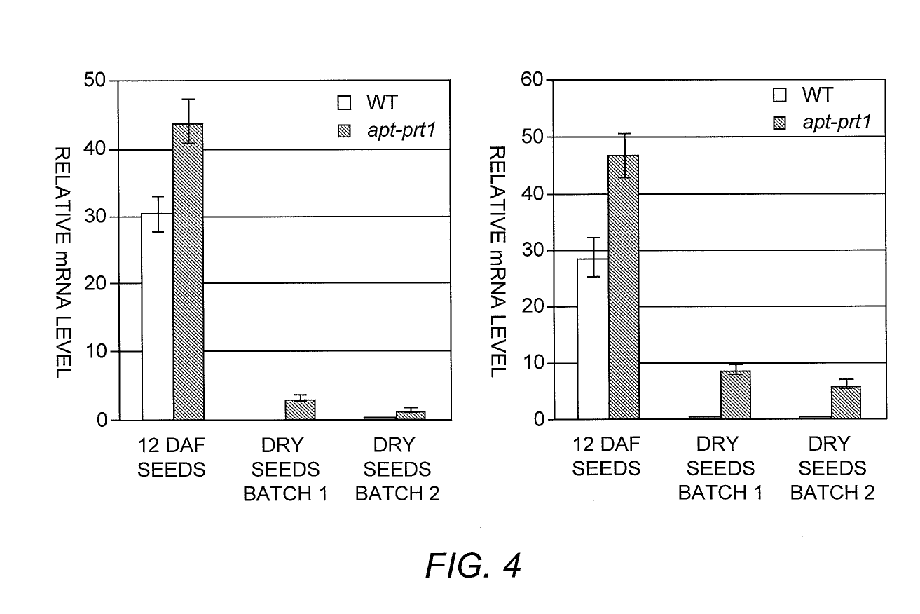

The elevated protein content in the atp-prt1-1 seeds was unexpected. To further analyze the altered protein-to-oil ratio, soluble sugars, organic acids, and free amino acids were measured in mature atp-prt1-1 seeds (FIG. 3). Levels of sucrose and glucose were elevated but the total amount of hexose was reduced in the atp-prt1-1 seeds (FIG. 3A). Moreover, the impaired conversion of sucrose and glucose into other hexose is indicative of a regulatory event inhibiting the mobilization of sucrose and glucose. Despite the increase of total seed protein, the free amino acids in atp-prt1-1 seeds were only about 50% of wild-type levels (FIG. 3A). Thus, the downstream protein sink strength in the mutant was increased, either due to an enhanced translational activity or an elevated storage and late embryogenesis abundant (LEA) protein gene expression. Consistent with both reduction of sugar and enhancement of amino acid utilization, the amount of TCA cycle metabolites was reduced. Taken together, the atp-prt1-1 seeds exhibit a reduced carbon flow from sucrose to oil and elevated protein sink strength.

The metabolic profiles of 15-day old seedlings were also compared between mutant and wild-type. Despite differences in tissue physiology, sugar profiles showed similar trends in seeds and seedlings, e.g., sucrose level increased and hexose level decreased, but the change of glucose was intermediate of the two (FIGS. 3B and 3C), indicating a down-regulation of sucrose and glucose utilization. Because soluble metabolites do not directly reflect fatty acid metabolic activities, the expression of fatty acid degradation enzymes in 2-week old atp-prt1-1 mutant seedlings was analyzed by RT-PCR. Genes encoding five peroxisomal fatty acid P-oxidation enzymes including the 3-ketoacyl-CoA thiolase gene PED1, the fatty acid multifunctional protein genes MFP2 and AIM1, the acyl-CoA oxidase gene ACX2, and a short chain acyl CoA oxidase gene At3g51840 were analyzed. Three out of the five genes, namely MFP2, AIM1, and ACX2 were reproducibly over-expressed in the atp-prt1-1 mutant. A fructose-6-phosphate 1-phosphotransferase P subunit gene At4g04040 and ubiquitin 10 (UBQ10) gene were expressed at similar levels in mutant and wild-type seedlings. The influence of ATP-PRT1 and ATP-PRT2 overexpression on fatty acid metabolism was also analyzed. In addition to being over-expressed in the atp-prt1-1 mutant, both AIM1 and MFP2 were over-expressed in ATP-PRT1 and ATP-PRT2 transgenic plants, a response likely due to the increased rate of fatty acid production.

The expression of ATP-PRT1 and ATP-PRT2 genes were also analyzed in wild-type and the atp-prt1-1 mutant. The elevated expression of ATP-PRT1 in mutant shoots and roots was a consequence of attenuated negative feedback by histidine (Ohta, et al. (2000) supra), wherein ATP-PRT2 appeared to be less sensitive to the feedback regulation as an increase in expression was not observed for ATP-PRT2 in the atp-prt1-1 mutant.

Alteration of mRNA levels of the fatty acid β-oxidation genes in the atp-prt1-1 mutant and the known effect of histidine on amino acid synthetic gene transcripts (Guyer, et al. (1995) supra) indicated that histidine and ATP-PRT1 exert a broad effect on regulating the transcriptional program. Thus, it was determined which genes regulated by ATP-PRT1 were correlated with ATP-PRT1 in their expression patterns during seed development. Using the publicly available Gene Correlator tool, Pearson's correlation coefficients were determined for ATP-PRT1 and genes expressed in developing seeds and siliques (ATGENEXPRESS® AFFYMETRIX® chips). Fatty acid metabolic genes were analyzed for comparison. Expression of cruciferin and albumin storage protein transcripts and LEA protein transcripts were all negatively correlated with ATP-PRT1. Among the large number of genes involved in the fatty acid synthesis, the Ketoacyl-ACP Synthase I (KAS I) gene showed a positive correlation and several fatty acid β-oxidation genes showed a negative correlation with ATP-PRT1 (Table 1). These results agree with the opposite changes of oil and protein contents in atp-prt1-1 mutant seeds and the detected overexpression of fatty acid β-oxidation genes in atp-prt1-1 shoots. Moreover, these data indicate that ATP-PRT1 coordinates cellular pathways at the transcriptional level.

TABLE 1

| | | |
|---|---|---|
| | Locus | Correlation |
| Transcript | Designation | Coefficient |

Fatty Acid Synthesis and Degradation

| | | |
|---|---|---|
| KAS I | At5g64290 | 0.581 |
| Acyl-CoA Oxidase | At5g65110 | −0.454 |
| | At3g51840 | −0.73 |
| Ketoacyl-CoA Thiolase | At2g33150 | −0.595 |
| | At5g48880 | 0.62 |
| Multifunctional Protein | At3g15290 | −0.476 |
| | At3g06860 | −0.562 |
| | At4g29010 | −0.369 |

Storage and LEA Proteins

| | | |
|---|---|---|
| CRA1 | At5g44120 | −0.563 |
| CRB | At1g03880 | −0.68 |
| CRU1 | At4g28520 | −0.641 |
| At2S1 | At4g27140 | −0.732 |
| At2S2 | At4g27150 | −0.605 |
| At2S3 | At4g27160 | −0.621 |
| At2S4 | At4g27170 | −0.678 |
| LEA | At1g52690 | −0.81 |
| | At2g44060 | −0.629 |
| | At2g36640 | −0.843 |
| | At5g44310 | −0.82 |
| | At3g22490 | −0.59 |

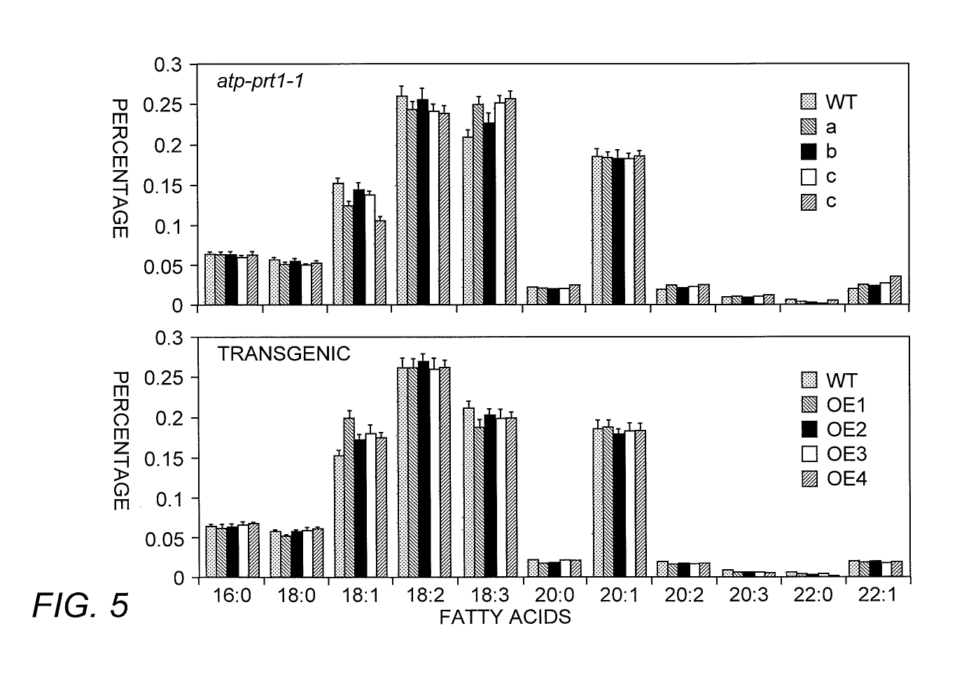

By way of further illustration, RT-PCR analysis of storage protein gene expression in 12 DAF developing seeds and mature dry seeds was conducted. The data in FIG. 5 shows that the overexpression of storage protein genes, At2S2 and At2S4, in a atp-prt1-1 mutant contributes to the increased protein sink strength and the reduced oil to protein ratio. These results indicate that histidine or ATP-PRT1 modify seed oil to protein ratio partly through regulation of storage protein gene expression.

The heterogeneity of fatty acids in plant tissues reflects an equilibration between the de novo fatty acid synthesis in plastids and the further chain elongation and desaturation in endoplasmic reticulum. In Arabidopsis, the 18 carbon stearic acid (18:0) and oleic acid (18:1) and the 16 carbon palmitic acid (16:0) are the three major fatty acids synthesized from plastids. They are converted to acyl-CoA upon leaving the plastids. If the plastidial acyl-CoA supply is reduced, the equilibrium shifts to desaturation and elongation. On the other hand, a higher rate of acyl-CoA production shifts the equilibrium to the opposite direction.

Significantly, the atp-prt1-1 mutant and ATP-PRT1 over-expressing transgenic lines exhibit opposing effects on fatty acid composition. Consistent with a reduced rate of carbon flow from sugar to plastidial fatty acids, atp-prt1-1 mutant seeds have an increase in 18:3 but reduction of 18:1 fatty acids. By contrast, a reduction of 18:3 but increase of 18:1 fatty acids in the transgenic seeds indicates an increased flow of carbon into fatty acids (FIG. 5). Moreover, the alteration of fatty acid flux also explains the opposite effects of chain elongation (from 18 into 20 and 22 carbons) caused by the mutant atp-prt1-1 and the transgene.

Phenotypically, transgenic plants that over-express ATP-PRT1 and ATP-PRT2 under the control of the 35S promoter exhibited similar phenotypes, indicating that the two isoforms have similar biochemical functions, although their physiological roles may differ because of different expression patterns. Leaves of both transgenic plants exhibited dark green areas surrounding the veins, indicative of increased chlorophyll content. In this regard, the Arabidopsis Co-Response database revealed a high correlation of ATP-PRT2 and photosynthetic gene expression. Thus, it is believed that ATP-PRT induces photosynthesis by signaling plants via a high nitrogen status.

Perturbation of development and growth was also observed in other tissues of transgenic plants. The flower organs petal, stamen, and carpel had impaired elongation. Within the carpel, ovules often occupied additional rows, resulting in a “fat” appearance of the siliques due to increased number of seeds inside. More frequent organogenesis and reduced tissue elongation was also reflected by the architecture of inflorescence, which produced more flowers with a smaller distance in between. Thus, it is believed that high nitrogen status mediates new organ initiation and tissue growth.

Taken together, multiple and concerted metabolic changes occur in the atp-prt1-1 mutant and cause reduced carbon assimilation and oil accumulation, but increased production of seed protein. Not wishing to be bound by a particular theory, it is believed that one or more of the following processes or genes is responsible for oil and protein content changes in the mutant seeds. Processes causing oil reduction may include sugar metabolism, glycolysis, fatty acid synthesis and degradation, and triacylglycerol synthesis. Processes affecting protein sink strength may include storage and LEA protein gene transcription or translation, genes contributing to the production of protein translational machinery, including RNA polymerase I and III, which are responsible for the transcription of rRNA and tRNA molecules, rRNA and tRNA processing enzymes, eukaryotic translational initiation and elongation factors (eIFs and eEFs), and ribosomal subunit genes.

Having demonstrated that ATP-PRT specifically functions to globally regulate carbon and nitrogen metabolism, the present invention is a method for altering oil or protein content of a plant or seed by modulating the expression or activity of ATP-phosphoribosyl transferase (ATP-PRT). As used in the context of the present invention, the terms altering or altered are intended to mean that oil and/or protein content in the plant of the instant method is increased or decreased as compared to a wild-type plant. Likewise, the terms modulating or modulated are intended to encompass increasing or decreasing expression or activity of ATP-PRT. In particular embodiments, decreasing the expression or activity of ATP-PRT results in an increase in protein content and a decrease in oil content, whereas increasing the expression or activity of ATP-PRT results in a decrease in protein content and an increase in oil content. In other embodiments, oil composition is altered by modulating the expression or activity of ATP-PRT.

Expression of ATP-PRT can be decreased or increased by generating plants expressing a mutant ATP-PRT protein or overexpressing a wild-type ATP-PRT protein, respectively, as exemplified herein. In one embodiment, ATP-PRT expression or activity is modulated in the whole plant. In another embodiment, expression in regulated by a constitutive plant promoter (e.g., CMV 35S promoter, rice actin, and maize ubiquitin). In a further embodiment, expression is regulated by an inducible plant promoter (e.g., cis-Jasmone promoter). In still another embodiment, tissue-specific expression of mutant or wild-type ATP-PRT is achieved using plant tissue-specific promoters such as seed-specific promoters, e.g., beta-conglycinin (Chiera, et al. (2004) Plant Mol. Biol. 56(6):895-904); leaf-specific promoters (Yamakawa, et al. (2004) J. Biosci. Bioeng. 98(2):140-3; Lin, et al. (2004) DNA Seq. 15(4):269-76); or root-specific promoters (Koyama, et al. (2005) J. Biosci. Bioeng. 99(1) :38-42), thereby altering tissue-selective carbon and nitrogen metabolism. Those skilled in the art can readily construct expression vectors and design protocols for recombinant gene expression. Suitable expression vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press. As used in the context of the present invention, the term operably linked is intended to mean that the ATP-PRT nucleic acid molecule is situated relative to the plant promoter such that transcription of the ATP-PRT message is regulated by the plant promoter. Many known techniques and protocols for manipulation of nucleic acid, for example in preparation of nucleic acid constructs, mutagenesis, sequencing, introduction of DNA into plant cells and gene expression, and analysis of proteins, are described in detail in Current Protocols in Molecular Biology, Second Edition, Ausubel et al. eds., John Wiley & Sons, 1992. Specific procedures and expression vectors previously used with wide success upon plants are described by Bevan (1984) Nucl. Acids Res. 12, 8711-8721; Guerineau and Mullineaux (1993) In: Plant Molecular Biology Labfax, Croy RRD ed., Oxford, BIOS Scientific Publishers, pp. 121-148; Miao and Lam (1995) Plant J. 7:359-365; Huang and Pan (2005) J. Agric. Food Chem. 53(10):3833-9; Chilton and Que (2003) Plant Physiol. 133(3):956-65; Iida and Terada (2005) Plant Mol. Biol. 59(1) :205-19). In particular, methods for transforming monocots are disclosed in U.S. Pat. No. 7,102,056, whereas methods for transforming dicots are disclosed for example in U.S. Pat. No. 7,112,721, the contents of which are incorporated herein by reference in their entireties.

Alternatively, ATP-PRT expression can be modulated using agents such RNA interference molecules (see, e.g., McGinnis, et al. (2005) Methods Enzymol. 392:1-24) or small molecule modulators. Likewise, ATP-PRT activity can be modulated using small molecule inhibitors or activators. See, e.g., Gohda, et al. (2001) Quant. Struct. Act. Relat. 20:143-14.

As the present invention also provides transgenic plants, progeny, seeds, and vegetatively reproducible structures from transgenic plants which express reduced or elevated levels of ATP-PRT as compared to wild-type plants, methods for using the same to identify inhibitors or activators of ATP-PRT gene expression or activity are also contemplated.

Plants embraced by the instant invention include crop plants such as rice, maize, wheat, barley, oats, rye, rape, potato, tomato, sugar beet, sunflower, soybean, and sorghum. ATP-PRT proteins, and nucleic acid sequences encoding the same, are well-known in the art for selected plant species (see, e.g., Arabidopsis ATP-PRT, GENBANK Accession Nos. NP_{—}563853 or NP_{—}176105; maize ATP-PRT, GENBANK Accession No. AY112299; wheat ATP-PRT, GENBANK Accession No. BT009385; sorghum ATP-PRT, GENBANK Accession No. CN139810; barley ATP-PRT, GENBANK Accession No. AJ485188). Alternatively, ATP-PRT nucleic acids can be readily identified using standard high stringency hybridization, e.g., a hybridization carried out in a solution of 5XSSPE (final 0.9 M NaCl, 0.05 M sodium phosphate, 0.005 M EDTA, pH 7.7), 5×Denhardt's solution, 0.5% SDS, at 50° C. to 65° C. overnight. Moreover, mutant ATP-PRT proteins are readily produced according to standard methods such as targeting induced local lesions in genomes, also known as TILLING (Till, et al. (2004) BMC Plant Biology 4:12; Till, et al. (2003) Methods Mol Biol. 236:205-20).

The invention is described in greater detail by the following non-limiting examples.

### EXAMPLE 1

### Microarray Analysis

To identify genes whose expression levels or timing patterns change in the atp-prt1-1 seeds, time course studies on the seed transcriptome are conducted. To trace the age of the siliques, flowers are labeled with colored thread on the day of pollination. Because the mutant plants are smaller and yield fewer seeds, a minimum number of time points are used to ensure a sufficient amount of material for each point. In addition to developing seeds, wild-type and mutant seedlings are be compared in microarray experiments. There are two reasons for using seedlings in the global analysis: the physiological roles of histidine and ATP-PRT1 in shoots, roots, and seeds are likely to differ, yet be related such that conclusions can be derived by integrating results from different tissues; and by adding histidine to the growth medium, the transient (and likely direct) responses to histidine are better characterized.

Wild-type and mutant seedlings are grown for 2 weeks without histidine, and then induced by histidine for a short period of time. The exact length of the induction period is determined by experimentally measuring the respond of genes that are known to be differentially expressed in atp-prt1-1 mutant and wild-type seedlings. With one biological duplicate for each experiment, a total of 32 AFFYMETRIX® ATH1 chips are needed to measure gene expression in siliques (five days after fertilization), seeds (8, 12, and 15 days after fertilization), shoots (±histidine) and roots (±histidine) for both wild-type and atp-prt1-1 mutant seedlings.

Total RNA from shoot and root tissues is isolated using a commercially available TRIZOL® kit. Extraction of seed RNA is according to standard methods (Parcy, et al. (1994) Plant Cell 6:1567-1582). PolyA RNA amplification, target labeling, hybridization, and microarray image scanning are conducted according to standard procedures.

Statistical analysis, including data transformation, normalization, and identification of differentially expressed genes using ANOVA model, is carried out using standard methods using a probe level analysis procedure (Chu, et al. (2002) Mathematical Bioscience 176:35-51).

### EXAMPLE 2

### Metabolite Profiling

Limited amounts of seed materials were used in the metabolite profiling analysis disclosed herein, and only those relatively abundant metabolites were detected. Profiling with a larger quantity of mutant materials provides a more complete view of the altered biochemical pathways in the atp-prt1-1 mutant. In addition, monitoring the dynamic change of metabolites during development and upon histidine induction offers insights into the behavior of the biochemical pathways in the atp-prt1-1 mutant and wild-type seedlings, and defines pathways that respond directly to histidine and ATP-PRT1.

Extraction of soluble metabolites and gas chromatography—mass spectrometry (GC-MS) determination of sugars, sugar alcohols, and organic acids is according to established protocols (Fiehn, et al. (2000) Anal. Chem. 72:3573-3580; Fiehn, et al. (2000) Nat. Biotechnol. 18:1157-1161; Roessner, et al. (2000) Plant J. 23:131-142). Free amino acids are measured using routine HPLC methodology (Brotherton, et al. (1996) Plant Cell Physiol. 37:389-394; Cho, et al. (2001) Plant Physiol. 123:1069-1076).

### EXAMPLE 3

### Cellular Pathways Downstream of ATP-PRT1 and Histidine

Genes that are differentially expressed in mutant, wild-type, and transgenic plants under at least one condition are clustered according to their expression patterns in mutant, wild-type, and transgenic plants across developmental stages and growth conditions. This is carried out using self-organizing map, K-mean, or hierarchical clustering algorithms in, e.g., GENESPRING™ software. In this regard, the major gene expression patterns that are preferentially influenced by ATP-PRT1 and histidine become evident and provide insights into the structure of the histidine-regulated transcriptional network, e.g., genes showing similar patterns and being influenced by atp-prt1-1 are likely positioned near each other in the network. It is expected that genes will fall into the following groups: genes showing opposite changes in the mutant and transgenic plants; genes affected in only mutant or transgenic lines; genes showing similar trends in mutant and transgenic lines. Each of the above three groups can be divided into histidine-dependent and -independent groups, and further divided according to more subtle differences in expression patterns. For example, genes showing opposite change in the mutant and transgenic plants are expected to be specifically downstream of ATP-PRT1.

To understand the functional pathways that are affected by ATP-PRT1, data from microarray and metabolite profiling studies are analyzed in MAPMAN, a bioinformatics tool that displays large data sets onto diagrams of metabolic pathways or other processes (Trimm, et al. (2004) Plant J. 37;907-914). This analysis reveals how various carbon metabolic pathways are affected by ATP-PRT1. Other affected processes such as nitrogen assimilation, amino acid metabolism, hormone synthesis and transport, purine metabolism, gene transcription and translation machinery, signaling pathways, etc. are also identified.

### EXAMPLE 4

### Protoplast System for Identification of Immediate Targets of ATP-PRT1 and Histidine

To explore the molecular mechanisms of ATP-PRT1 and histidine regulation of gene expression, a cell-based system is employed. Protoplasts are desirable for this purpose because genes and metabolites are conveniently introduced into protoplasts and their immediate targets are identified. To determine whether ATP-PRT1 and histidine modulate gene expression in protoplasts as they do in intact plants, RT-PCR is conducted to test gene expression after introduction of ATP-PRT1 and histidine into mesophyll protoplasts.

The effector construct is prepared by cloning the ATP-PRT1 cDNA into pWPF143 between the 35S promoter and the NOS 3′ UTR. This vector is a pUC19-based and the size is relatively small. Protoplast preparation and transformation is according to standard methods. Total RNA is isolated from protoplasts using commercially available TRIZOL® reagent and RT-PCR is conducted to determine the expression of candidate downstream genes. Approximately 100,000 protoplasts yields 2 pg of RNA, which is enough for at least 50 RT-PCR analyses. Histidine is also introduced directly into protoplasts following the protoplast transformation protocol, wherein histidine is used in place of the effector DNA.

### EXAMPLE 5

### Time Course Analysis of Storage Accumulation During atp-prt1-1 Seed Development

To monitor the rate of oil and protein accumulation at particular seed developmental stages, time course analysis of storage accumulation is conducted. Seeds of ages ranging from 5 to 20 day-after-fertilization are harvested and used for storage product measurement. Transient starch is also measured to obtain a comprehensive view of the storage process in the atp-prt1-1 mutant. Methods of seed oil and starch quantification are known in the art (Lin, et al. (2004) Plant Physiol. 135(2):814-27). For the determination of seed protein content, total proteins are extracted in 0.5% SDS before measurement of proteins in the supernatant. These results are indicative of altered storage processes. For instance, change of gene expression prior to or correlated with oil reduction in time reveals a chemical step that is responsible for the decreased oil content.

### EXAMPLE 6

### Identifying Biochemical Steps in Oil Content Reduction and Protein Content Increase

To identify the steps involved in regulation of oil and protein metabolism, expression time course of genes throughout the period of seed development are compared between the mutant and the wild-type by real time RT-PCR. Real time RT-PCR is conducted using TAQMAN® assay kit and the ABI 7900HT Real-Time PCR system, following manufacture's instruction (Applied Biosystems, Foster city, Calif.). Enzymes catalyzing altered biochemical steps are subsequently assayed for activities according to established methods (Focks and Benning (1998) Plant Physiol. 118:91-101; Hutchings, et al. (2005) Exp. Bot. 56:577-585; Hill, et al. (2003) Plant Physiol. 131:228). If alteration of enzyme activity is not accompanied by change of mRNA at a given step, western blot analysis is conducted to measure the protein level of the corresponding gene. Metabolite feeding experiments are also conducted (Focks and Benning (1998) supra; Lin, et al. (2004) supra). By feeding developing seeds radiolabeled metabolites and measuring the rate of label incorporation into oil and protein, it is determined whether a particular intermediate is metabolized at a normal rate to synthesize oil or protein. Moreover, to measure tRNA and rRNA components of the translational machinery, total tRNA and rRNA are quantified and RT-PCR measurement of specific tRNA and rRNA genes is conducted. Because the total RNA is composed of polyA RNA, tRNA, and rRNA, yield of polyA RNA from total RNA in the mutant and the wild-type seeds is compared.

### EXAMPLE 7

### Regulatory Functions of ATP-PRT

To confirm that alteration of gene expression, metabolism, and oil to protein ratio in atp-prt1-1 mutant were specific consequences of the ATP-PRT1 loss-of-function mutation and not, secondary effects, ATP-PRT1 was over-expressed in transgenic Arabidopsis to demonstrate opposing phenotypes. To demonstrate that the control of histidine flow by ATP-PRT1 is essential in balancing metabolic pathways and coordinating seed oil and protein accumulation, point mutations that alter the catalytic or allosteric functions of ATP-PRT1 are introduced. It is expected that histidine will be produced at elevated or reduced levels under a given carbon and energy status. In other words, carbon and energy status will be under- or over-reflected by the nitrogen signal histidine, causing altered balancing between cellular pathways and between protein and oil production processes.

For over-expression of ATP-PRT1 and ATP-PRT2, the ATP-PRT1 and ATP-PRT2 cDNAs were individually cloned into pCambia1301 between the 35S promoter and the NOS 3′ UTR. T-DNA containing the 35S::ATP-PRT1::NOS3′ or 35S::ATP-PRT2::NOS3′ cassette was transformed into Arabidopsis using the flower dip method (Clough and Bent (1998) Plant J. 16:735-743). Independent transgenic lines were tested by RT-PCR for the expression of ATP-PRT1 or ATP-PRT2 and histidine-regulated genes. The seed oil composition of ATP-PRT1 transgenic lines was also determined.

Amino acid residues that make specific contacts with PRPP, AMP, and histidine in the ATP-PRT1 protein are deduced from that of the E. coli homolog (Lohkamp, et al. (2004) supra). Introduction of point mutations at these conserved residues is expected to interfere with PRPP, AMP, or histidine binding, and result in attenuated or enhanced enzymatic activity.

To obtain at least two ATP-PRT1 mutants with elevated and reduced activities, respectively, various mutant forms are tested in bacteria to determine activities. For example, three mutant forms, namely Arg127Ala to attenuate PRPP binding, Gly86Ala and Arg87Ala to attenuate AMP binding, and Pro342Ala and Thr343Ala to attenuate histidine binding, are analyzed. Additional mutants are generated via PCR mutagenesis (Kammann, et al. (1989) Nucl. Acid Res. 17:5404) using point mutations included in the PCR primers. Mutant and wild-type cDNAs without the N-terminal transit peptide region are cloned into the pET19b E. coli. T7 expression vector (EMD Biosciences, Darmstadt, Germany) and recombinant proteins are affinity purified under native condition using HisBind purification.

ATP-PRT activity is conducted according to established methods (Martin (1963) J. Biol. Chem. 238:257-268) with the high extinction coefficient of PR-ATP at 290 nM. Briefly, reaction mixture containing 80 mM Tris-HCl (pH 8.5), 4 mM ATP, 8 mM MgCl₂, and recombinant ATP-PRT is measured for blank absorption at 290 nM before addition of 0.3 mM PRPP and measurement of PR-ATP production at 290 nM.

To further associate ATP-PRT1 mutant forms with seed oil and protein contents and gene expression, mutant cDNAs of desired properties and wild-type cDNA are expressed in null T-DNA insertional mutant plants. To avoid complication caused by ectopic gene expression, the ATP-PRT1 promoter is used. The DNA constructs are made by replacing the 35S promoter in the pCambia1301 with the promoter of ATP-PRT1, and subsequent insertion of mutant or wild-type cDNA between the ATP-PRT1 promoter and the NOS 3′ UTR. Several independent transgenic lines can be obtained for each construct. These transgenic lines are measured for seed oil and protein contents and for expression of representative genes. Enzymes with enhanced and reduced activities are expected to confer opposite effects on both gene expression and seed oil to protein ratio.

### EXAMPLE 8

### Chlorophyll Content and Photosynthetic Rates of ATP-PRT Transgenic Plants

The atp-prt1-1 mutant was pale green in comparison to wild-type. Although the phenotype indicates a role of ATP-PRT1 in chlorophyll accumulation or chloroplast development, a non-specific effect on growth deficiency is possible. To determine whether over-expression of ATP-PRT stimulates chlorophyll accumulation, chlorophyll content is measured in different ATP-PRT transgenic lines and wild-type. Chlorophyll a and b and carotenoid are extracted with 95% ethanol and determined by measuring absorbance at 470, 648.6, and 664.2 nm minus absorbance at 730 nm. Calculation of pigment content is as described in the art (Lichtenthaler (1987) Methods Enzymol. 148:350-382). The expression of photosynthetic genes is revealed by microarray analysis.

What is claimed is:

1. A transgenic plant with reduced or elevated levels of ATP-phosphoribosyl transferase expression or activity, wherein at least one tissue of the transgenic plant exhibits altered protein or oil content compared to wild-type.

2. A progeny of the transgenic plant of claim 1.

3. A seed of the transgenic plant of claim 1.

4. A vegetatively reproducible structure of the transgenic plant of claim 1.

5. An expression vector comprising a nucleic acid molecule encoding a wild-type or mutant ATP-phosphoribosyl transferase operably linked to a plant promoter.

6. A method for altering the protein or oil content of at least one tissue of a plant comprising introducing the expression vector of claim 5 into a plant so that the expression or activity of ATP-phosphoribosyl transferase in at least one tissue of the plant is modulated thereby altering the protein or oil content.

7. A method for altering the protein or oil content of at least one tissue of a plant comprising modulating the expression or activity of ATP-phosphoribosyl transferase in at least one tissue of the plant by contacting the tissue with an agent which increases or decreases expression or activity of ATP-phosphoribosyl transferase so that protein or oil content of the tissue is altered.
